(19) United States
(12) Patent Application Publication
(10) Pub. No.: US 2024/0268843 A1
(43) Pub. Date: Aug. 15, 2024

(54) SYSTEM FOR TREATING THROMBUS IN BODY LUMENS

(71) Applicant: Shockwave Medical, Inc., Santa Clara, CA (US)

(72) Inventor: Hoa NGUYEN, Santa Clara, CA (US)

(73) Assignee: Shockwave Medical, Inc., Santa Clara, CA (US)

(21) Appl. No.: 18/642,498

(22) Filed: Apr. 22, 2024

Related U.S. Application Data
(63) Continuation of application No. 17/477,483, filed on Sep. 16, 2021, now Pat. No. 11,992,232.
(60) Provisional application No. 63/106,275, filed on Oct. 27, 2020.

(51) Int. Cl. A61B 17/22

(52) U.S. Cl. CPC A61B 17/22012; A61B 2017/22007; A61B 2017/22021; A61B 2017/22079

(57) ABSTRACT

The present disclosure relates generally to thrombectomy devices. An exemplary catheter comprises: a central tube; an emitter assembly mounted over the central tube, wherein the emitter assembly comprises: a conductive sheath; a first insulated wire having a first curved distal portion; and a second insulated wire having a second curved distal portion, wherein the first curved distal portion and the second curved distal portion are positioned within the conductive sheath, and wherein the emitter assembly is configured to generate a plurality of cavitation bubbles or shockwaves when a pulsed voltage is applied to the emitter assembly; and an outer tube housing the emitter assembly, wherein the outer tube is configured to receive a conductive fluid, wherein the outer tube comprises a distal opening for releasing the plurality of cavitation bubbles or shockwaves and the conductive fluid in a forward direction to treat thrombus at a treatment site.

### PRIOIRTY

This application is a continuation of U.S. patent application Ser. No. 17/477,483, filed Sep. 16, 2021, which claims priority to U.S. Provisional Patent Application Ser. No. 63/106,275, filed Oct. 27, 2020, the entire disclosure of each of which is incorporated by reference.

### FIELD OF THE DISCLOSURE

The present disclosure relates generally to thrombectomy devices, and more specifically, to thrombectomy devices designed to generate cavitation bubbles and/or shockwaves for reducing or removing thrombus from the vascular system of a patient.

### BACKGROUND

Thrombectomy devices are designed to reduce clot burden and partially or completely remove a blood clot (i.e., thrombus) from the vascular system of a patient. Currently, the mechanism of removing thrombus in most thrombectomy devices is mechanical or involves a combination of the plasminogen activator (“tPA”) treatment and a mechanical process. Some of these devices use ultrasound for the purpose of diffusing tissue plasminogen activator (tPA). It does this by increasing permeability in thrombus structure, which exposes more sites to which thrombolytic agents can bind. These devices all have deficiencies, as they provide an undesirably slow rate of clot removal, which typically requires an overnight stay in the hospital. Further, these devices tend to be expensive, bulky, and difficult to operate. Further still, these devices can involve a high loss of blood in the patient.

Accordingly, a need exists for a device that treats blood clots without the use of drugs (e.g., tPA) and provides a cost-effective and time-efficient solution for treating thrombus.

### BRIEF SUMMARY

The present invention relates to thrombectomy devices designed to generate cavitation bubbles and/or shockwaves for reducing or removing thrombus from the vascular system of a patient. Because embodiments of the present invention do not require the use of drugs (e.g., tPA) and can work quickly (e.g., less than 2 hours), the present invention provides a cost-effective and efficient solution for treating thrombus. Short procedure time can minimize bleeding complication and reduce infection.

An exemplary catheter comprises: a central tube; an emitter assembly mounted over the central tube, wherein the emitter assembly comprises: a conductive sheath; a first insulated wire having a first curved distal portion; and a second insulated wire having a second curved distal portion, wherein the first curved distal portion and the second curved distal portion are positioned within the conductive sheath, and wherein the emitter assembly is configured to generate a plurality of cavitation bubbles or shockwaves when a pulsed voltage is applied to the emitter assembly; and an outer tube housing the emitter assembly, wherein the outer tube is configured to receive a conductive fluid, wherein the outer tube comprises a distal opening for releasing the plurality of cavitation bubbles or shockwaves and the conductive fluid in a forward direction to treat thrombus at a treatment site.

In some embodiments, when the pulsed voltage is applied across the first wire and the second wire, a current is configured to: flow from the first wire to the conductive sheath to generate a first plurality of cavitation bubbles or shockwaves and, flow from the conductive sheath to the second wire to generate a second plurality of cavitation bubbles or shockwaves.

In some embodiments, the first wire comprises a first exposed distal tip and the second wire comprises a second exposed distal tip.

In some embodiments, the current is configured to flow from the first exposed tip to the conductive sheath and from the conductive sheath to the second exposed tip via a conductive fluid.

In some embodiments, the current is configured to flow from the first exposed distal tip to an inner wall and/or front edge of the conductive sheath.

In some embodiments, the central tube comprises a first segment and a second segment closer to a proximal end of the central tube than the first segment, where the diameter of the second segment is larger than the first segment.

In some embodiments, the second segment comprises a longitudinal groove for accommodating a part of the first insulated wire.

In some embodiments, the curved distal portion of the first wire wraps around the first segment of the central tube.

In some embodiments, the central tube comprises a central lumen for accommodating a guide wire.

In some embodiments, a pump is configured to deliver a continuous flow of conductive fluid to the emitter assembly through the outer tube.

In some embodiments, the conductive fluid comprises saline.

In some embodiments, the continuous flow of conductive fluid flushes debris away from the treatment site.

In some embodiments, the voltage is between 800V and 2700V.

In some embodiments, a repetition rate of the applied voltage pulses is between approximately 4 Hz and 100 Hz.

In some embodiments, the electrode pair comprises a spark gap between electrodes of the pair, the spark gap being less 0.005″.

### DESCRIPTION OF THE FIGURES

FIG. 1A depicts an exemplary emitter assembly that can be used in a forward firing intravascular catheter, in accordance with some embodiments.

FIG. 1B depicts wires of the exemplary emitter assembly, in accordance with some embodiments.

FIG. 2A depicts parts of an exemplary forward firing intravascular catheter, in accordance with some embodiments.

FIG. 2B depicts parts of the exemplary forward firing intravascular catheter, in accordance with some embodiments.

FIG. 2C depicts parts of the exemplary forward firing intravascular catheter, in accordance with some embodiments.

FIG. 2D depicts parts of the exemplary forward firing intravascular catheter, in accordance with some embodiments.

FIG. 3A depicts a cross-sectional view of the distal end of the exemplary forward firing intravascular catheter, in accordance with some embodiments.

FIG. 3B depicts a front view of the distal end of the exemplary forward firing intravascular catheter, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Described herein are exemplary systems and methods for reducing or removing thrombus from the vascular system of a patient by generating cavitation bubbles and/or shockwaves via a voltage source. In accordance with some embodiments, the treatment system includes a forward firing intravascular catheter that releases cavitation bubbles, shockwaves, or a combination thereof. The catheter comprises a central tube for housing a guidewire, an emitter assembly that can be mounted over the central tube, and an outer tube. In some embodiments, the distal end of the outer tube is open (as opposed to sealed), thus allowing cavitation bubbles and/or shockwaves created by the emitter assembly to be released in a forward direction via the distal opening of the outer tube.

In operation, the catheter can be advanced within the body lumen (e.g., a blood vessel) to the treatment site (e.g., via a guide wire). The emitter assembly includes electrodes that, when connected to pulse generator, form plasma arcs that in turn lead to the generation of shockwaves and/or cavitation bubbles. The cavitation bubbles and/or shockwaves are released in a forward direction via the distal opening of the outer tube. The cavitation bubbles and/or shockwaves create mechanical vibrations, turbulence, jets, and/or forceful collapses to weaken and break the fibrin fiber network, thus reducing and removing thrombus.

In the preferred embodiment, the present invention is essentially a hybrid system combining aspects of both shock wave generation and cavitation bubble generation. The voltage at each emitter (i.e., across the spark gap) can be somewhat lower than conventional intravascular lithotripsy (“IVL”). In addition, the repetition rate is somewhat higher than a typical IVL treatment so as to produce both shock waves and cavitation bubbles. In some embodiments, the catheter can be powered by an 825D generator available from Shockwave Medical, Inc. (IVL Generator Catalog Number IVLGCCD). In some embodiments, the voltage of the generator is adjusted between 1800V and 2700V, and the repetition rate is adjusted to between approximately 4 Hz and 25 Hz. In order to maintain the breakdown voltage, the spark gap at the emitter (e.g., a spark gap formed between two electrodes of an electrode pair) is sufficiently small to allow a spark. In some embodiments, the gap is less than 0.005 inches.

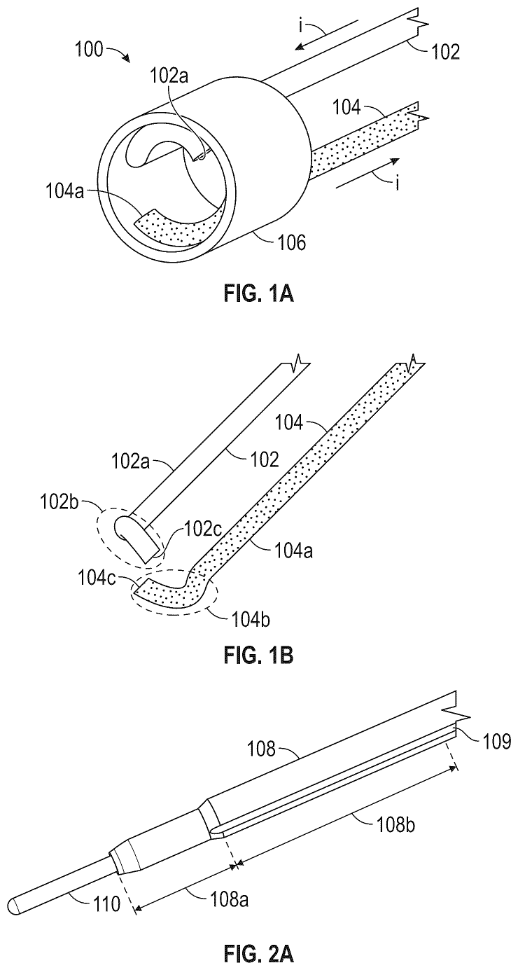

FIG. 1A depicts an exemplary emitter assembly 100 that can be used in a forward firing intravascular catheter, in accordance with some embodiments. The emitter assembly 100 comprises a first wire 102, a second wire 104, and a conductive sheath 106. In some embodiments, the conductive sheath can be stainless steel hypotubes, and the wires can be polyimide-insulated copper wires.

FIG. 1B provides a perspective view of the wires 102 and 104, in accordance with some embodiments. Wire 102 comprises a straight portion 102a, a curved distal portion 102b, and a conductive distal end 102c. In some embodiments, the conductive distal end 102c is formed by cutting the wires 102 to expose the conductive core under the insulation material. Similarly, wire 104 comprises a straight portion 104a, a curved portion 104b, and a conductive distal end 104c.

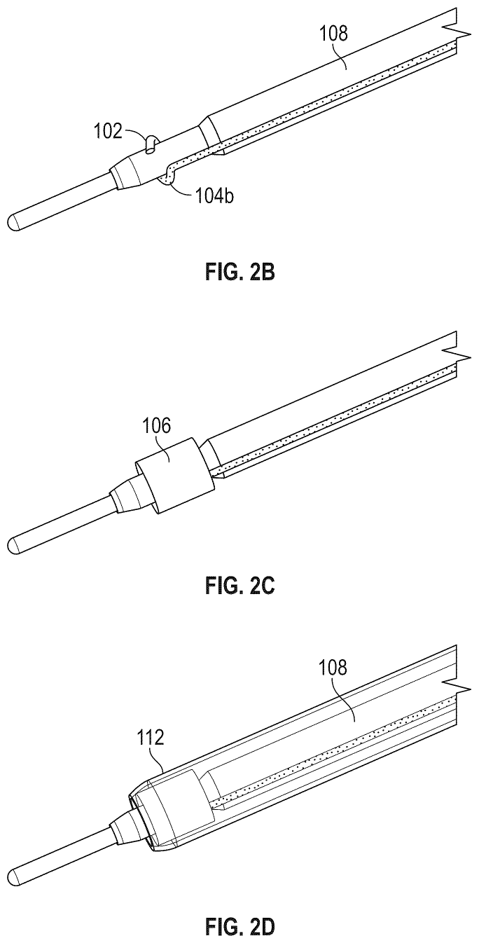

The emitter assembly can be mounted over a central tube within a catheter. With reference to FIG. 2A, an exemplary central tube 108 comprises a central lumen for accommodating a guidewire 110. The central tube further comprises longitudinal grooves (e.g., groove 109) on its outer surface for accommodating wires of the emitter assembly.

In the depicted example in FIG. 2A, the central tube 108 comprises a segment 108a and a segment 108b, where the diameter of the segment 108b is larger than 108a. One or more longitudinal grooves (e.g., groove 109) can be formed over the segment 108b to accommodate wires of the emitter assembly. The deepest point in the groove in segment 108b can be aligned with the outer surface of the segment 108a such that a wire can extend through the groove in the segment 108b and also rest over the segment 108a in a flat or substantially flat manner, as illustrated by wire 104 in FIG. 2B.

FIGS. 2B and 2C illustrate how an emitter assembly (e.g., the emitter assembly of FIG. 1A) can be mounted over the central tube 108. With reference to FIG. 2B, the wire 104 extends through a longitudinal groove of the central tube 108 and its distal curved portion 104b wraps around the central tube. The wire 102 can be mounted in a similar manner through another longitudinal groove of the central tube 108. The wires can be bonded to the outer surface of the central tube with adhesive (e.g., epoxy or cyanoacrylate adhesive). With reference to FIG. 2C, the conductive sheath 106 of the emitter assembly is placed over (without touching) the distal curved portions of the wires 102 and 104. The conductive sheath 106 can be secured in place with adhesive (e.g., epoxy or cyanoacrylate adhesive). There is a gap between the conductive sheath and each of the distal ends of wires 102 and 104. In some embodiments, the gap is between approximately 0.003″ to 0.006″.

With reference to FIG. 2D, the catheter further comprises an outer tube 112. The outer tube 112 opens at the distal end. As shown, the outer tube 112 wraps around the emitter assembly, including the conductive sheath and the wires. The outer tube can be made of a non-conductive polymer or elastomer (e.g. polyester, polyurethane, polyimide, neoprene, silicone).

Turning back to FIG. 1A, in operation, when the emitter assembly 100 is connected to a voltage source, a current i traverses through the first insulated wire 102, the conductive sheath 106, and the second insulated wire 104 to generate cavitation bubbles and/or shockwaves at two locations. Specifically, the proximal end of the wire 102 is connected to a positive port of a voltage generator (not depicted) and the proximal end of the wire 104 is connected to a negative port of the voltage generator. The generator delivers energy in continuous pulse mode or in the series of short bursts. Accordingly, the current i traverses the emitter assembly as indicated by the arrows. As shown, the current, i, traverses from the proximal end of the wire 102 toward its distal curved portion. At the distal end of the wire 102, the conductive core of the wire is exposed, thus allowing the current to traverse the gap between the distal end of the wire 102 and the conductive sheath 106 (e.g., the inner wall and/or the front edge of the conductive sheath) via a conductive fluid. The exposed distal end of the wire 102 and the conductive sheath 106 form a first electrode pair for generating cavitation bubbles and/or shockwaves.

The current i further traverses the gap between the conductive sheath 106 and the distal end of the second insulated wire 104. At the distal end of the second insulated wire 104, the conductive core of the wire is exposed, thus allowing the current to traverse the gap between the conductive sheath 106 (e.g., the inner wall and/or the front edge of the conductive sheath) and the conductive distal end of the wire 104 via the conductive fluid. The exposed distal end of the second wire 104 and the conductive sheath 106 form a second electrode pair for generating cavitation bubbles and/or shockwaves. The current i then returns to the voltage generator via the second insulated wire 104.

As the current i traverses between a wire and the conductive sheath, a plurality of plasma arcs are formed between the exposed distal end of the wire and the inner wall and/or the front edge of the conductive sheath. Plasma arcs lead to cavitation bubbles and/or shockwaves in a controlled fashion (one at a time, at a particular rate), which in turn lead to mechanical vibrations, and other bubble dynamics-related effects such as collapses, turbulence, jetting, etc. in the conductive fluid (e.g., via the expansion and collapse of the bubbles). The mechanical vibrations serve to reduce or remove the thrombus. Cavitation has been known to weaken the fibrin network crosslink which is the base structure of the thrombus. Combination of mechanical vibrations and bubble cavitation can be effective in thrombolysis.

As shown in FIG. 1A, each of the distal curved portions of the wires 102 and 104 is curved in the same direction as the adjacent inner wall of the conductive sheath 106. In some embodiments, the distal curved portion of each wire can be parallel or substantially parallel to the adjacent inner wall of the conducive sheath. As the plasma arcs cause erosion to the electrodes, each curved portion can erode and shorten over time. Because the curvature of the curved wire portion is similar (e.g., in the same direction) or identical to the curvature of the adjacent inner wall of the conductive sheath, the shortest gap between the distal end of the wire and the conductive sheath can be kept constant or substantially constant, thus ensuring that cavitation bubbles and/or shockwaves are generated in a constant or substantially constant manner. Note that as the curved wire portion erodes, the location of the generation of the cavitation bubbles and/or shockwaves will change. In the illustrated embodiment, the location of the generation of the cavity bubbles and/or shockwaves will rotate circumferentially about the inner periphery of the conductive sheath 106.

As compared to the generators used in the prior art shock wave generation systems mentioned above, the generator for this system may be configured to generate lower-voltage pulses at a higher pulse repetition rate in order to obtain the benefits of both cavitation bubbles and shock waves. For example, in the prior art systems, each pulse might be about 3000 volts with a 1 Hz repetition rate. In embodiments of this system, voltage is adjusted between 800V to 2700V; repetition rate is adjusted to approximately 4 Hz and 100 Hz; pulse duty cycle is adjusted between 20%-60%. These parameters can be varied based on the blood clot condition.

Additional details on electrode pairs formed by a wire and a conductive tube, along with possible variations, are provided in assignee's prior filings U.S. Publication No. 2019/0388110 titled “SYSTEM FOR TREATING OCCLUSIONS IN BODY LUMENS” and U.S. Publication No. 2021/0085348, titled “SYSTEM FOR TREATING THROMBUS IN BODY LUMENS,” both of which are incorporated by reference. It should be appreciated that, while FIGS. 1A-2D depict a emitter assembly comprising two wires and a conductive sheath driven by one voltage source, other configurations of emitter assembly can be placed at the distal tip of the catheter to provide forward-firing capabilities.

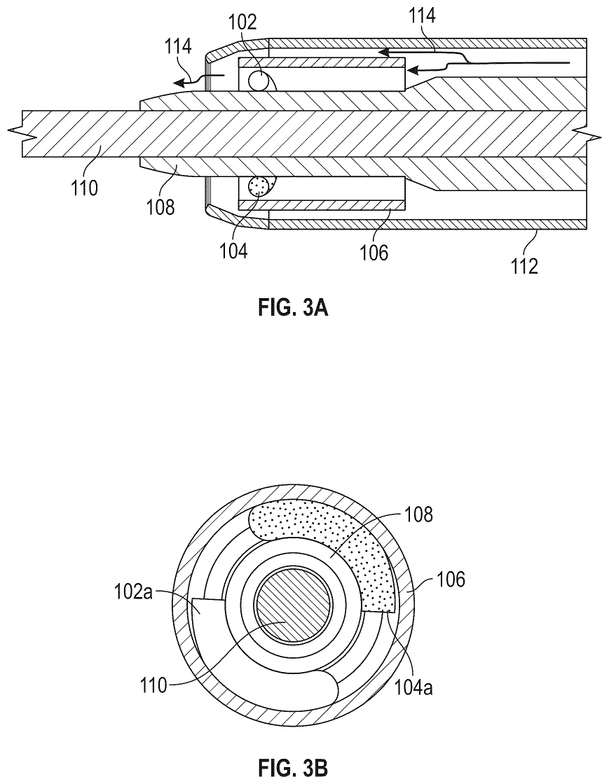

FIGS. 3A and 3B depict a cross-sectional view and a front view of the distal end of the exemplary forward firing intravascular catheter, respectively, in accordance with some embodiments. The central tube 108 comprises a central lumen for accommodating a guidewire 110. An emitter assembly, which comprises wires 102 and 104 and a conductive sheath 106, is mounted over the central tube 108. The emitter assembly comprises two emitters and each emitter comprises an electrode pair. A first electrode pair comprises the distal end of wire 102 and the conductive sheath 106, and a second electrode pair comprises the distal end of the wire 104 and the conductive sheath 106. As seen in FIG. 3B, the curved distal potions have a coiled configuration. Each curved potion extends in a range from about 50 to 120 degrees about the circumference of the first segment 108a of the central tube.

The catheter further comprises an outer tube 112, which wraps around and covers the emitter assembly. In operation, the outer tube 112 is used to deliver an ionic solution (e.g., a conductive solution such as saline or saline mixed with a contrast medium) from a pump to the emitter assembly. When the emitter assembly is connected to a voltage source, plasma arcs are formed via the conductive fluid at each electrode pair (e.g., between the distal end of each wire and the sheath's inner wall and/or the front edge), which in turn lead to a large amount of cavitation bubbles forming and collapsing and/or shockwaves, as described above. The flow of conductive fluid can be continually administered to transport the cavitation bubbles and/or shockwaves forward.

The outer tube 112 has a distal opening for releasing the cavitation bubbles in a forward direction. The electrode pairs are placed close to the distal opening to maximize the release of cavitation bubbles. As indicated by arrows 114 in FIG. 3A, the cavitation bubbles from the emitter assembly are carried out in the forward direction by the pumped flow of the ionic solution through the distal opening to the thrombus. Further, shockwaves are propagated forward through the distal opening. The pumped flow further removes debris (e.g., metals, bubbles) and thrombus fragments from the treatment site. The rapid removal of debris helps to refresh the cavitation. In some embodiments, the pump provides both infusion and aspiration.

The catheter can be used in conjunction with a pump. In some embodiments, the pump delivers an ionic solution (i.e., a conductive solution such as saline or saline mixed with a contrast medium) via the outer tube to the catheter tip where the cavitation takes place. The pump or an auxiliary pump also aspirates debris away from the thrombus region. The infusion flow can be synchronized to the emitter assembly's power delivery to ensure the adequate ionic solution around the emitters. The aspiration flow and infusion flow can be synchronized to maintain the pressure equilibrium at the treatment site. In some examples, the flow of saline or saline/angiographic contrast medium is adjusted to avoid over-heating issues and control treatment efficiency and rate.

In some embodiments, additional components are included in the treatment system, such as a proximal balloon for trapping debris produced by the emitter, a visualization system and/or a steering system for properly navigating (e.g., side branches) and placing the catheter, etc. Additional details of the treatment system are provided in US Publication Nos. 2019/0388110, and 2021/0085348, referenced above and incorporated herein by reference.

In some embodiments, the procedure can take around 30 minutes to an hour, during which the emitter assembly continuously generates cavitation bubbles and/or shockwaves. These operation parameters (e.g., voltage, repetition rate, or pulse duty cycle of the voltage pulses) can be set based on the characteristics of the blood clot (e.g., size of the clot, age of the clot, composition of the clot, softness of the clot, arterial or venous location of the clot, platelet content of the clot, fibrin content of the clot, or some other attribute of the clot) and/or characteristics of the patient (e.g., age or preexisting medical condition of the patient). In some embodiments, after the procedure, a post-operation minimally invasive procedure (e.g., treatment of bleeding, thrombus reforming) can be performed.

It will be understood that the foregoing is only illustrative of the principles of the invention, and that various modifications, alterations and combinations can be made by those skilled in the art without departing from the scope and spirit of the invention. Any of the variations of the various cavitation devices disclosed herein can include features described by any other cavitation devices or combination of shock wave devices herein. Furthermore, any of the methods can be used with any of the cavitation devices disclosed. Accordingly, it is not intended that the invention be limited, except as by the appended claims. For all of the variations described above, the steps of the methods need not be performed sequentially

1. A catheter comprising:
a central tube;
an emitter assembly mounted over the central tube, wherein the emitter assembly comprises:
a conductive sheath;
a first insulated wire; and
a second insulated wire,
wherein a distal portion of the first insulated wire and a distal portion of the second insulated wire are positioned within the conductive sheath, and
wherein the emitter assembly is configured to generate a plurality of cavitation bubbles or shock waves when a pulsed voltage is applied to the emitter assembly; and
an outer tube housing the emitter assembly,
wherein the outer tube is configured to receive a conductive fluid,
wherein the outer tube comprises a distal opening for releasing the plurality of cavitation bubbles or shock waves and the conductive fluid in a forward direction to treat thrombus at a treatment site.

2. The catheter of claim 1, wherein, when the pulsed voltage is applied across the first insulated wire and the second insulated wire, a current is configured to:
flow from the first insulated wire to the conductive sheath to generate a first plurality of cavitation bubbles or shock waves and,
flow from the conductive sheath to the second insulated wire to generate a second plurality of cavitation bubbles or shock waves.

3. The catheter of claim 2, wherein the first insulated wire comprises a first exposed distal tip and the second insulated wire comprises a second exposed distal tip.

4. The catheter of claim 3, wherein the current is configured to flow from the first exposed tip to the conductive sheath and from the conductive sheath to the second exposed tip via a conductive fluid.

5. The catheter of claim 4, wherein the current is configured to flow from the first exposed distal tip to an inner wall and/or front edge of the conductive sheath.

6. The catheter of claim 2, wherein the central tube comprises a first segment and a second segment closer to a proximal end of the central tube than the first segment, where the diameter of the second segment is larger than the first segment.

7. The catheter of claim 6, wherein the second segment comprises a longitudinal groove for accommodating a part of the first insulated wire.

8. The catheter of claim 6, wherein the distal portion of the first insulated wire wraps around the first segment of the central tube.

9. The catheter of claim 1, wherein the central tube comprises a central lumen for accommodating a guide wire.

10. The catheter of claim 1, wherein a pump is configured to deliver a continuous flow of conductive fluid to the emitter assembly through the outer tube.

11. The catheter of claim 10, wherein the conductive fluid comprises saline.

12. The catheter of claim 10, wherein the continuous flow of conductive fluid flushes debris away from the treatment site.

13. The catheter of claim 1, wherein the voltage is between 800V and 2700V.

14. The catheter of claim 1, wherein a repetition rate of the applied voltage pulses is between approximately 4 Hz and 100 Hz.

15. The catheter of claim 1, wherein the emitter assembly comprises a first spark gap between a distal end of the first insulated wire and the conductive sheath and a second spark gap between a distal end of the second insulated wire and the conductive sheath, wherein the first and second spark gap are between 0.003 inches and 0.006 inches.

16. The catheter of claim 15, wherein the first spark gap is less than 0.005 inches.

17. A catheter for treating a thrombus in a blood vessel comprising:
an elongated central member;
a first insulated wire extending along the central member, wherein the distal tip of the first wire is not insulated;
a second insulated wire extending along the central member, wherein the distal tip of the second wire is not insulated;
a cylindrical conductive shield positioned around and spaced from the distal ends of the first and second insulated wires; and
an outer tube surrounding the cylindrical conductive shield and having an open distal end configured so that conductive fluid injected into the proximal end of the outer tube will pass over the distal tips of the first and second insulated wires and be directed out the distal end of the outer tube towards the thrombus and wherein when high voltage pulses are applied across the first and second insulated wires, current will flow along the first insulated wire and jump a first gap between the distal tip of the first insulated wire to the cylindrical conductive shield and then along the cylindrical conductive shield and then jump a second gap between the cylindrical conductive shield and the distal tip of the second insulated wire and back along the second insulated wire to create, at both the first and second gaps, cavitation bubbles or shock waves that are forwardly directed toward the thrombus.

18. The catheter of claim 17, wherein the central member includes a lumen for accommodating a guide wire.

19. The catheter of claim 17, wherein the voltage of the high voltage pulses is between 800V and 2700V.

20. The catheter of claim 17, wherein a repetition rate of the applied high voltage pulses is between approximately 4 Hz and 100 Hz.

21. The catheter of claim 17, wherein the first and second gaps are both between 0.003 inches and 0.006 inches.

22. The catheter of claim 17, wherein the central member comprises a first segment and a second segment closer to a proximal end of the central member than the first segment, where the diameter of the second segment is larger than the first segment.

23. The catheter of claim 22, wherein the second segment comprises a longitudinal groove for accommodating a part of the first insulated wire.

24. The catheter of claim 22, wherein a distal portion of the first insulated wire wraps around the first segment of the central member.

25. The catheter of claim 17, wherein a pump is configured to deliver a continuous flow of conductive fluid into the proximal end of the outer tube.

26. The catheter of claim 25, wherein the conductive fluid comprises saline.

27. The catheter of claim 25, wherein the continuous flow of conductive fluid flushes debris away from the thrombus.

28. The catheter of claim 17, wherein the first gap is less than 0.005 inches.

29. A catheter for treating a thrombus in a blood vessel comprising:
an elongated central member;
a first insulated wire extending along the central member and electrically connected at its distal end to a first electrode pair;
a second insulated wire extending along the central member and electrically connected at its distal end to a second electrode pair;
a cylindrical conductive shield positioned around and spaced from the distal ends of the first and second insulated wires, wherein the cylindrical conductive shield forms an electrode of the first electrode pair and an electrode of the second electrode pair; and
an outer tube surrounding the cylindrical conductive shield and having an open distal end configured so that conductive fluid injected into the proximal end of the outer tube will pass over the distal tips of the first and second insulated wires and be directed out the distal end of the outer tube towards the thrombus and wherein when high voltage pulses are applied across the first and second insulated wires, current will flow along the first insulated wire and jump a first gap of the first electrode pair and then along the cylindrical conductive shield and then jump a second gap of the second electrode pair and back along the second insulated wire to create, at both the first and second gaps, cavitation bubbles or shock waves that are forwardly directed toward the thrombus.

30. The catheter of claim 29, wherein the central member includes a lumen for accommodating a guide wire.

31. The catheter of claim 29, wherein the voltage of the high voltage pulses is between 800V and 2700V.

32. The catheter of claim 29, wherein a repetition rate of the applied high voltage pulses is between approximately 4 Hz and 100 Hz.

33. The catheter of claim 29, wherein the first and second gaps are both between 0.003 inches and 0.006 inches.

34. The catheter of claim 29, wherein the first gap is less than 0.005 inches.

35. The catheter of claim 29, wherein the central member comprises a first segment and a second segment closer to a proximal end of the central member than the first segment, where the diameter of the second segment is larger than the first segment.

36. The catheter of claim 35, wherein the second segment comprises a longitudinal groove for accommodating a part of the first insulated wire.

37. The catheter of claim 35, wherein a distal portion of the first insulated wire wraps around the first segment of the central member.
